(19) United States
(12) Patent Application Publication
(10) Pub. No.: US 2014/0119495 A1
(43) Pub. Date: May 1, 2014

(54) RADIATION IMAGING APPARATUS AND METHOD FOR RECONSTRUCTING IMAGE

(71) Applicant: SAMSUNG ELECTRONICS CO., LTD., Suwon-si (KR)

(72) Inventors: Kwang Eun JANG, Seoul (KR); Jong Ha LEE, Hwaseong-si (KR); Ji Young CHOI, Suwon-si (KR); Young Hun SUNG, Hwaseong-si (KR)

(73) Assignee: SAMSUNG ELECTRONICS CO., LTD., Suwon-si (KR)

(21) Appl. No.: 14/065,809

(22) Filed: Oct. 29, 2013

Related U.S. Application Data
(60) Provisional application No. 61/719,904, filed on Oct. 29, 2012.

(30) Foreign Application Priority Data
Jul. 25, 2013 (KR) 10-2013-0088035

(51) Int. Cl. A61B 5/00

(52) U.S. Cl. CPC A61B 5/72
USPC 378/4

(57) ABSTRACT

Disclosed are an image reconstruction method and a radiation imaging apparatus using the same. The radiation imaging apparatus includes a radiation emitter which emits multiple energy spectra of radiation toward an object, a radiation detector which detects multiple energy spectra of radiation passing through the object and thereby outputs measurement data, and an image reconstructor which reconstructs a radiation image of the object, based on the measurement data output by the radiation detector. The method may be used to calculate simulation data of an inner structure of the object using an image reconstruction value including information associated with densities of substances of the inside of the object, to acquire a correction value of the image reconstruction value minimizing a discrepancy between the measurement data and the simulation data and to update the image reconstruction value using the correction value.

### CROSS-REFERENCE TO RELATED APPLICATIONS

This application claims the benefit of U.S. Provisional Patent Application No. 61/719,904, filed on Oct. 29, 2012 in the U.S. Patent and Trademark Office, and priority from Korean Patent Application No. 10-2013-0088035, filed on Jul. 25, 2013 in the Korean Intellectual Property Office, the disclosures of which are incorporated herein by reference in their respective entireties.

### BACKGROUND

1. Field

Exemplary embodiments relate to an image reconstruction method and an image reconstruction apparatus for reconstructing an image.

2. Description of the Related Art

An imaging apparatus collects various outside information and reconstructs and produces a predetermined image which corresponds to the collected information, based on the collected information. Visible light, infrared light, radioactive light, ultrasonic waves, microwaves and the like may be used as various outside information. The imaging apparatus may be classified into various categories, depending on type of outside information. For example, imaging apparatuses include cameras, infrared cameras, radiation imaging apparatuses, ultrasonic wave imaging apparatuses, radars and the like.

A radiation imaging apparatus is an imaging system for acquiring an image of an object by emitting radiation toward the object. The object may be a human or an article such as baggage. In addition, the radiation may be an X-ray.

Such a radiation imaging apparatus is widely used in a variety of fields, such as medical diagnosis in medicine, baggage inspection in airports, or checking of inner structures of objects or parts in industrial or construction sites due to advantage of easily finding an inner structure of the object, without destroying the object.

Examples of the radiation imaging apparatus include digital radiography (DR) apparatuses, fluoroscopy apparatuses, cardiography apparatuses, computed tomography (CT) apparatuses, mammography apparatuses and the like.

### SUMMARY

Therefore, it is one aspect of one or more exemplary embodiments to provide a radiation imaging apparatus and an image reconstruction method which reconstruct a radiation image in which more substances than the number of types of energy spectra of radiations emitted toward an object are distinguished from one another.

It is another aspect of one or more exemplary embodiments to provide a radiation imaging apparatus and an image reconstruction method which reconstruct a radiation image having a high accuracy, without deterioration in image quality in the process of image reconstruction using radiation imaging.

It is another aspect of one or more exemplary embodiments to provide a radiation imaging apparatus and an image reconstruction method which clearly distinguish bones from contrast agents.

In order to solve these aspects, an image reconstruction method and a radiation imaging apparatus are provided.

In accordance with one aspect of one or more exemplary embodiments, an image reconstruction method includes emitting K-energy spectra of radiation toward an object and detecting K-energy spectra of radiation which propagate through the object in order to acquire measurement data which relates an inside of the object, initializing an image reconstruction value, acquiring a correction value which relates to the image reconstruction value which correction value reduces a discrepancy between the measurement data and simulation data, wherein the simulation data includes data which is associated with an inside structure of the object which data is acquirable by using the initialized image reconstruction value, and updating the image reconstruction value by using the acquired correction value, wherein the image reconstruction value relates to information which is associated with K+α substances of the inside of the object, wherein each of K and α is a respective natural number.

In accordance with another aspect of one or more exemplary embodiments, a radiation imaging apparatus includes a radiation emitter which is configured to emit multiple energy spectra of radiation toward an object, a radiation detector which is configured to detect multiple energy spectra of radiation which propagate through the object and to output measurement data based on the detected multiple energy spectra, and an image reconstructor which is configured to reconstruct a radiation image of the object, based on the outputted measurement data, wherein the image reconstructor is further configured to acquire a correction value which relates to an image reconstruction value which correction value reduces a discrepancy between the measurement data and simulation data, and to update the image reconstruction value by using the acquired correction value, wherein the simulation data includes data which relates to an inner structure of the object which is acquirable by using the image reconstruction value which relates to information which is associated with K+α substances of the inside of the object, wherein each of K and α is a respective natural number.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a view illustrating a configuration of a radiation imaging apparatus, according to an exemplary embodiment;

FIG. 2 is a view illustrating the radiation imaging apparatus, according to an exemplary embodiment;

FIG. 3 is a view illustrating an example of operations of a radiation emission unit and a radiation detection unit;

FIG. 4 is a view illustrating another example of the radiation emission unit;

FIG. 5 is a concept view illustrating an exemplary embodiment of the radiation emission unit;

FIG. 6 is a view illustrating emitted radiation;

FIG. 7 is a perspective view illustrating an exemplary embodiment of the radiation detection unit;

FIG. 8 is a graph illustrating an operation of an image reconstruction unit;

FIG. 9 is a block diagram illustrating an exemplary embodiment of the image reconstruction unit;

FIG. 10 is a flowchart illustrating an image reconstruction method according to an exemplary embodiment; and

FIG. 11 is a flowchart illustrating an image reconstruction method according to another exemplary embodiment.

### DETAILED DESCRIPTION

Reference will now be made in detail to exemplary embodiments, examples of which are illustrated in the accompanying drawings, wherein like reference numerals refer to the like elements throughout.

Hereinafter, a radiation imaging apparatus as an exemplary embodiment of an imaging apparatus which may be used for performing an image reconstruction method will be described. However, the radiation imaging apparatus using image reconstruction is not limited to the radiation imaging apparatus described below. Examples of the radiation imaging apparatus using image reconstruction include digital radiography apparatuses, fluoroscopy apparatus, cardiography apparatuses, computed tomography (CT) apparatuses and mammography apparatuses. In addition, the radiation imaging apparatus using image reconstruction may be applied to a variety of tomography apparatuses which use tomosynthesis such as dual energy computed tomography (CT) apparatuses and C-arm computed tomography (CT) apparatuses.

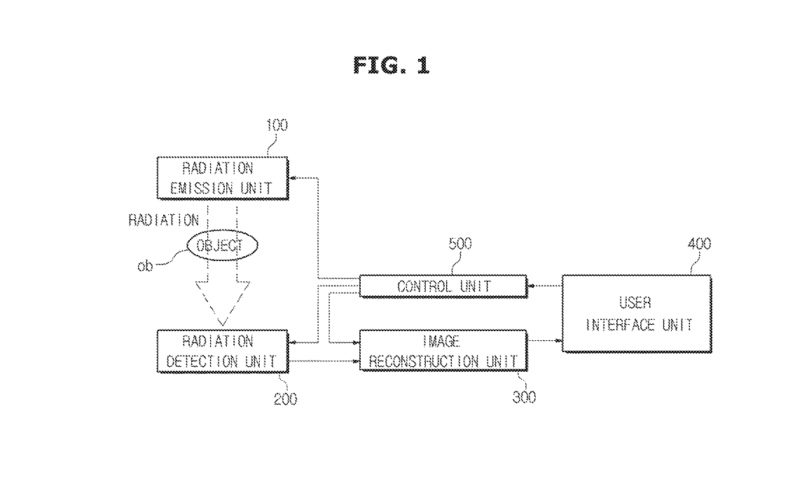

FIG. 1 is a view illustrating a configuration of a radiation imaging apparatus, according to an exemplary embodiment.

As shown in FIG. 1, the radiation imaging apparatus according to an exemplary embodiment includes a radiation emission unit (also referred to herein as a radiation emitter) 100, a radiation detection unit (also referred to herein as a radiation detector) 200, and an image reconstruction unit (also referred to herein as an image reconstructor) 300. The radiation imaging apparatus may further include a user interface unit (also referred to herein as a user interface) 400 and a control unit (also referred to herein as a controller) 500.

The radiation emission unit 100 generates a predetermined energy spectrum of radiation and emits the radiation toward an object (ob).

The radiation emission unit 100 may generate different spectra of radiation and emit the same toward the object (ob). The radiation emission unit 100 may generate radiation several times and emit the same toward the object (ob). In this case, during each radiation emission period, the radiation emission unit 100 may generate different spectra of radiation and emit the same toward the object (ob).

In one exemplary embodiment, the radiation emission unit 100 may emit a monochromatic energy spectrum of radiation toward the object (ob). In another exemplary embodiment, the radiation emission unit 100 may emit a polychromatic energy spectrum of radiation toward the object (ob).

In addition, the radiation emission unit 100 may simultaneously emit different spectra of radiation toward the object. In this case, the radiation emission unit 100 may include a plurality of radiation tubes which generate different radiations.

According to an exemplary embodiment, the radiation imaging apparatus may include one radiation emission unit 100 or a plurality of radiation emission units 100. In addition, the radiation emission unit 100 may be fixed or movable.

When radiation is emitted from the radiation emission unit 100 toward the object, the applied radiation is absorbed in or propagates through inner tissues of the object (ob) according to characteristics of the tissues. In this case, an attenuation coefficient is a value which indicates how much radiation is absorbed in or propagates through each tissue. Attenuation coefficients vary according to respective tissues. In addition, attenuation coefficients may vary according to an energy spectrum of the applied radiation.

Specifically, regarding the radiation imaging apparatus, an intensity of radiation which propagates through a predetermined tissue is determinable by applying the following Equation 1.

I=I₀e^{-μt} [Equation 1]

wherein I₀ represents an intensity of emitted radiation, and μ represents an intensity of radiation which propagates through an object (ob), μ represents an attenuation coefficient according to an inner tissue of the object (ob), and t represents a thickness of an inner tissue of the object (ob) through which radiation passes. As is seen from Equation 1, an attenuation of radiation increases as a thickness of the inner tissue of the object increases or as the attenuation coefficient increases.

The object (ob) may be a living thing such as human or animals, or a non-living thing such as baggage, equipment, or structure.

In an exemplary embodiment, a predetermined contrast agent may be injected into the object (ob). The contrast agent is a chemical which increases a contrast of inner substances when injected into human or animals so as to clearly distinguish inner substances, such as, for example, one tissues or blood vessels, from other tissues during radiographic inspection. More specifically, the contrast agent artificially increases or decreases a difference in radiation absorbance between inner substances of the object (ob), and thereby increases a corresponding contrast of the inner substances upon radiography, such as, for example, magnetic resonance imaging or computed tomography (CT). Accordingly, when the contrast agent is used, bio-structures or lesions are easily distinguished from the surroundings, and diagnosis of patients and the like are thus easier and more accurate.

One type of contrast agent or a plurality of contrast agents may be added to an object (ob). When the plurality of contrast agents are added to the object (ob), the radiation emission unit 100 emits a plurality of different spectra of radiation toward the object (ob) according to the number of types of added contrast agents.

Examples of the contrast agent include iodine, an iodine-gadolinium complex, barium sulfate (BaSO₄) and the like. Furthermore, gas such as carbonic acid gas may be used as the contrast agent.

The radiation, having propagated through the object (ob), may be detected by the radiation detection unit 200.

The radiation detection unit 200 receives the radiation emitted by the radiation emission unit 100 and outputs a predetermined electric signal corresponding to the received radiation. In this case, the radiation received by the radiation detection unit 200 may be radiation which propagates through the object (ob) and is attenuated by inner substances of the object (ob).

According to an exemplary embodiment, the radiation imaging apparatus may include one radiation detection unit 200 or a plurality of radiation detection units 200. In addition, the radiation detection unit 200 may be fixed or movable.

The predetermined electric signal output by the radiation detection unit 200 is subjected to processes, such as, for example, analog-to-digital (A/D) conversion or amplification, and is then transferred to the image reconstruction unit 200. Hereinafter, the electric signal transferred to the image reconstruction unit 200 is referred to as “measurement data”.

The image reconstruction unit 200 reconstructs a predetermined radiation image, based on the measurement data acquired by the radiation emission unit 100 and the radiation detection unit 200.

The user interface unit 400 displays the radiation image, and/or allows a user to input predetermined instructions, commands, and/or various data.

The control unit 500 controls the overall operation of the radiation imaging apparatus. For example, the control unit 500 controls a movement operation of at least one of the radiation emission unit 100 and the radiation detection unit 200, and/or an emission operation of the radiation emission unit 100. According to an exemplary embodiment, the control unit 500 controls movement of the radiation emission unit 100 and/or the radiation detection unit 200, emission of the radiation emission unit 100 or the like, according to the instruction or command of the user input via the user interface unit 400.

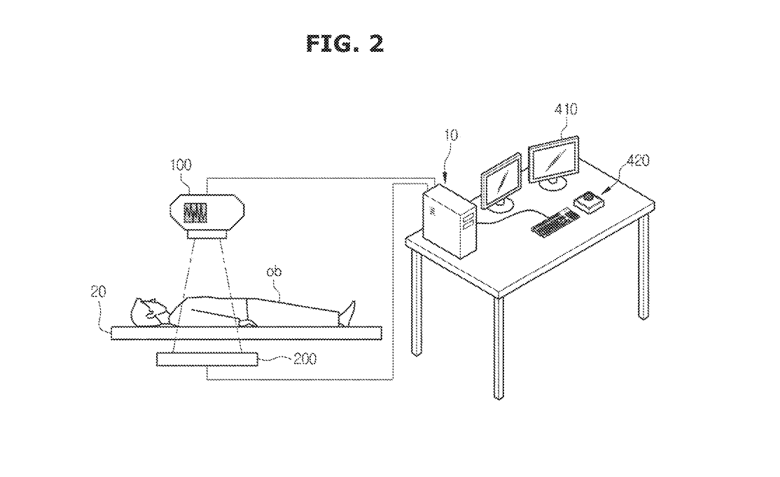

FIG. 2 is a view illustrating the radiation imaging apparatus, according to the present exemplary embodiment.

More specifically, as shown in FIG. 2, the radiation imaging apparatus may be a digital radiography apparatus which is configured to generate an image of an inner structure of a human or the like. A general radiation imaging apparatus may further include a stand 20, a console 10 and a user interface unit 400, in addition to the radiation emission unit 100 and the radiation detection unit 200.

The object (ob) may be supported by the stand 20. The stand 20 may be disposed between the radiation emission unit 100 and the radiation detection unit 200 such that radiation is emitted toward the object (ob) and radiation which has propagated through the object (ob) is received. The object (ob) may be a living thing, such as, for example, a human or animal, or a non-living thing, such as, for example, baggage.

The console 10 may be connected to at least one of the radiation emission unit 100 and the radiation detection unit 200 with or without wire, and may transmit and/or receive various control commands or data from at least one of the radiation emission unit 100 and the radiation detection unit 200. The console 10 may be provided with the image reconstruction unit 300 and the control unit 500 of the radiation imaging apparatus. According to an exemplary embodiment, the image reconstruction unit 300 or the control unit 500 may be provided in the radiation detection unit 200, instead of being provided in the console 10.

The user interface unit 400 may include a display unit (also referred to herein as a display) 410 and an input unit 420, as shown in FIG. 2.

The display unit 410 may display the radiation image reconstructed by the image reconstruction unit 300. For example, the display unit 410 may be a monitor device connected to the console 10 by wire or wirelessly. According to an exemplary embodiment, the display unit 410 may be a display device which is configured to display a general two-dimensional image or a three-dimensional image. The display unit 410 may be mounted in an exterior housing of the radiation emission unit 100, according to an exemplary embodiment.

The input unit 420 inputs predetermined information, instructions, and/or commands from operators of a radiation imaging apparatus, for example, doctors, radiologic technicians, nurses or patients. The input unit 420 transfers the input information, instructions, and/or commands to the control unit 500 provided in the console 10 or the like. The input unit 420 may be connected to the console 10 by wire or wirelessly in order to transfer the instruction, command, and/or information to the control unit 500 provided in the console 10.

According to an exemplary embodiment, the input unit 420 may be directly mounted on a partial module of the radiation imaging apparatus, for example, the radiation emission unit 100.

The input unit 420 may be, for example, at least one of buttons, keyboards, mice, track-balls, track-pads, levers, handles and sticks, or a combination of at least two thereof. In addition, the input unit 420 may be a touchscreen module including a touchscreen panel. When the input unit 420 is a touchscreen module, the input unit 420 may also function as the display unit 410.

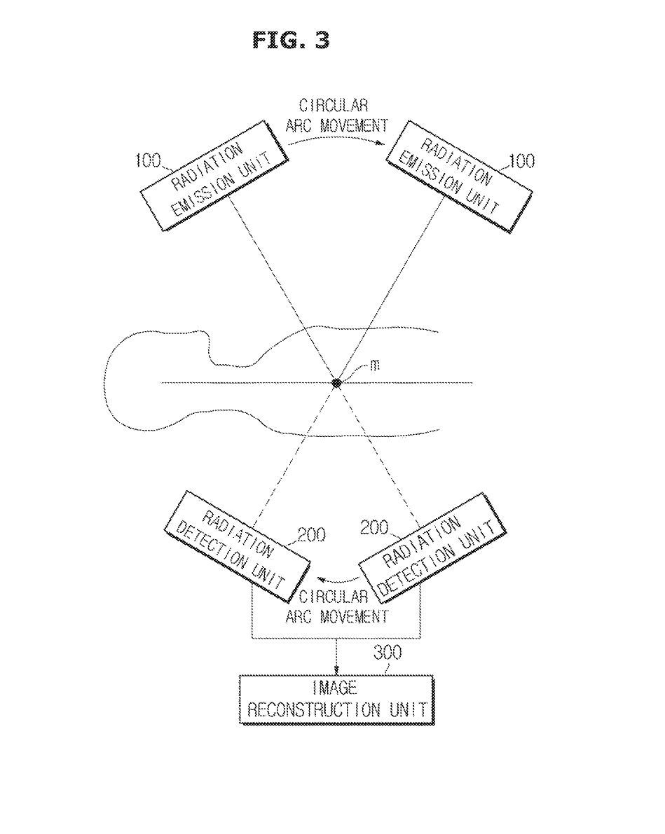

FIG. 3 is a view illustrating an example of operations of the radiation emission unit and the radiation detection unit.

As shown in FIG. 3, the radiation emission unit 100 may emit radiation toward the object (ob) while doing circular arc movement.

The radiation emission unit 100 emits a polychromatic energy spectrum of radiation or different spectra of radiation toward the object (ob) while moving in a circular arc. For example, when the radiation imaging apparatus is a computed tomography (CT) apparatus, the radiation emission unit 100 may simultaneously or separately emit a polychromatic energy spectrum of radiation or different spectra of radiation to the object (ob) while a gantry rotates once. In this case, according to an exemplary embodiment, the radiation emission unit 100 may emit different spectra of radiation toward the object (ob) whenever the gantry rotates.

In an exemplary embodiment, the radiation detection unit 200 may receive radiation from the radiation emission unit 100 while it is fixed. In another exemplary embodiment, as shown in FIG. 3, the radiation detection unit 200 may receive radiation from the radiation emission unit 100 while moving in a circular arc corresponding to the circular arc movement of the radiation emission unit 100.

Measurement data collected by the radiation detection unit 200 may be transferred to the image reconstruction unit 300.

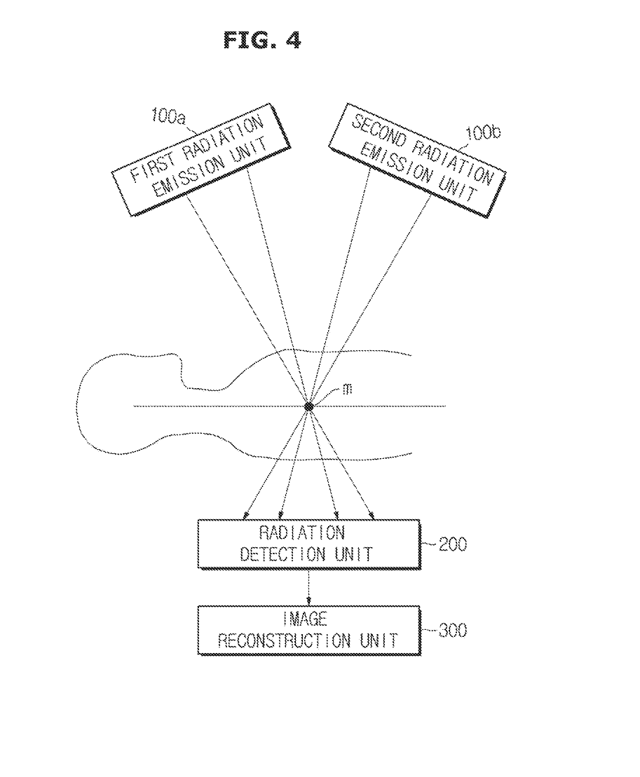

FIG. 4 is a view illustrating another example of operations of the radiation emission unit.

As shown in FIG. 4, a plurality of radiation emission units, for example, a first radiation emission unit 100a and a second radiation emission unit 100b, may be provided in the radiation imaging apparatus. In this case, respective radiation emission units 100a and 100b may emit different spectra of radiation toward the object (ob). At least one of the first radiation emission unit 100a and the second radiation emission unit 100b may travel while moving in a circular arc, as shown in FIG. 3. Both of the radiation emission units 100a and 100b may be may travel while moving in a circular arc.

In one exemplary embodiment, the radiation imaging apparatus may be provided with one radiation detection unit 200 which receives radiation from the plurality of radiation emission units 100a and 100b, as shown in FIG. 4. In another exemplary embodiment, the radiation imaging apparatus may be provided with a plurality of radiation detection units 200 corresponding to the respective radiation emission units 100a and 100b, and the plurality of radiation detection units 200 may respectively receive radiation from the radiation emission units 100a and 100b corresponding thereto.

The radiation detection unit 200 may receive radiation from the radiation emission unit 100 while it is fixed, or may receive radiation from the radiation emission unit 100 while moving in a circular arc corresponding to the circular arc movement of the radiation emission unit 100.

As described above, measurement data collected by the radiation detection unit 200 may be transferred to the image reconstruction unit 300.

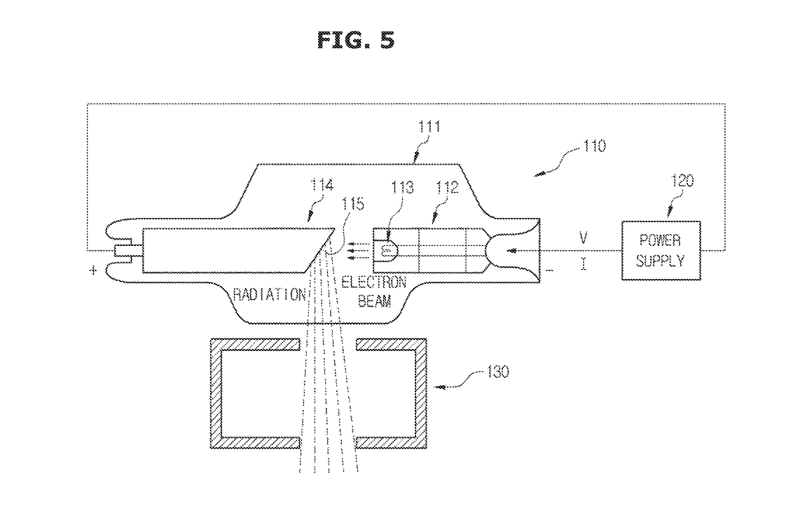

Hereinafter, one exemplary embodiment of the radiation emission unit 100 will be described in more detail. FIG. 5 is a schematic view illustrating an exemplary embodiment of the radiation emission unit.

As described above, the radiation emission unit 100 of the radiation imaging apparatus generates radiation with a predetermined energy and emits the radiation in a predetermined direction, for example, a direction of the object (ob). For this purpose, the radiation emission unit 100 may include a radiation tube 110 which is configured to generate radiation and a power supply 120 which is configured to apply a predetermined voltage and/or a predetermined current to the radiation tube 110, as shown in FIG. 5.

The radiation tube 110 may include a tube body 111, a cathode 112, and an anode 114.

The tube body 111 may include a variety of parts such as cathode 112 and an anode 114, and the cathode 112 and the anode 114 may be stably fixed in the tube body 111. In addition, the tube body 111 may prevent leakage of electrons which are generated in the cathode 112 and which move to the anode 114. A vacuum level of the tube body 111 may be maintained at a high value of about 10⁻⁷ mmHg. The tube body 111 may be, for example, a glass tube made of a predetermined hard silicate glass.

The cathode 112 may emit an electron beam including a plurality of electrons according to the predetermined voltage (V) and/or the predetermined current (I) which is applied from the power supply 120 in a direction of the anode 114.

Specifically, the cathode 112 may include a filament 113 which is configured to accumulate the electrons. The filament 113 of the cathode 112 may be connected to the power supply 120 and be heated according to the voltage applied from the power supply 120. The cathode 112 heated by the applied voltage emits electrons (an electron beam of the filament 113 into the tube body 111). In this case, an energy of the electrons may be determined according to tube voltage. In an exemplary embodiment, the filament 113 of the cathode may be made of tungsten (W). The cathode 112 may optionally include a focusing electrode which is configured to focus emitted electrons.

Further, according to an exemplary embodiment, the cathode 112 may include carbon nanotubes, instead of the filament 113. The electrons emitted from the filament 113 of the cathode 112 move toward the anode 114 while being accelerated in the tube body 111.

The anode 114 rapidly decelerates the accelerated electrons and generates predetermined radiation in accordance with the law of conservation of energy.

Specifically, the anode 114 may be provided with a target 115 which decelerates while colliding with the electrons generated in the filament 113. The accelerated electrons which move toward the anode 114 collide with the target 115 formed in the anode 114 and are rapidly decelerated by a coulomb force. When electrons are decelerated, radiation of an energy corresponding to the applied tube voltage in accordance with the law of conservation of energy is generated.

In one exemplary embodiment, the anode 114 may be a fixed anode, as shown in FIG. 5. The fixed anode 114 may be cut at a predetermined cutting angle, as shown in FIG. 5. The target 115 with which electrons which are emitted from the filament 113 and are accelerated collide may be formed in a cut portion of the fixed anode 114. In this case, for example, the cutting angle of the fixed anode 114 may be 20 degrees in a vertical direction, based on tube axis. The target 115 may be provided with a focal point, a plane of the target 115 with which the accelerated electrons collide. The focal point may have a rectangular shape. At the focal point, predetermined radiation is emitted in accordance with collision of the accelerated electrons.

The anode 114 may be made of a metal, such as, for example, copper, and the target 115 may be made of a metal such as, for example, at least one of tungsten (W), chromium (Cr), iron (Fe) or nickel (Ni).

In another exemplary embodiment, the anode may be a rotation anode having a rotatable circular plate shape (not shown). The rotation anode may rotate, based on the movement direction of the accelerated electrons as an axis. The rate of rotation of the rotation anode may, for example, be in the range from 3,600 RPM to 10,800 RPM. An end of the rotation anode, that is, an end of the circular plate, may be cut to a predetermined angle, similarly as the fixed anode. The target with which the electrons emitted from the filament 13 collide may be formed in the cut portion of the end of the rotation anode. As described above, the target 115 may be provided with a focal point, a plane of the target 115 with which the accelerated electrons collide. The rotation anode may be rotated by a rotor which is coupled to the rotation anode and the target formed in the cut portion of a boundary of the circular plate according to rotation of the rotation anode may also rotate, similarly as the rotation anode.

The rotation anode advantageously increases heat accumulation and decreases focal point size, as compared to the fixed anode. Furthermore, it may be possible to obtain a clearer radiation image.

Radiation generated from the anode 115 may be emitted toward the object (ob). In one exemplary embodiment, a collimator 130 which is configured to control a parameter, such as, for example, emission range of radiation, may be formed in an emission direction of the radiation.

The collimator 130 transmits radiation emitted in a predetermined direction and absorbs and/or reflects radiation emitted in directions excluding the predetermined direction, thereby filtering radiation. Accordingly, the collimator 130 enables the radiation emission unit 100 to emit radiation in a predetermined range and/or in a predetermined direction. The collimator 130 may be formed using a material to absorb radiation, such as, for example, lead (Pb).

According to an exemplary embodiment, the radiation which has propagated through the collimator 130 may further pass through a predetermined filter made of aluminum (Al) or copper (Cu). The radiation which has propagated through the collimator 130 may be attenuated while passing through the predetermined filter. As a result, a dose of radiation emitted toward the object (ob) may be reduced to some extent. The predetermined filter may change an energy spectrum of the emitted radiation. In this case, one filter may be used, or a combination of a plurality of filters having different energy spectra may be used.

The power supply 120 may apply a predetermined voltage (V) and/or a predetermined current (I) to the anode 114 and the cathode 112 of the radiation tube 110. A potential difference between the cathode filament 211 and the anode 222 of the radiation tube 22 is referred to as tube voltage, and current applied by electrons colliding with the anode 222 is referred to as tube current. As tube voltage increases, velocity of the electrons increases and an energy of generated radiation thus increases. As tube current increases, radiation dose may increase. Using these concepts, the power supply 120 controls applied voltage (V) and current and thereby controls energy spectrum and radiation dose emitted from the radiation emission unit 100.

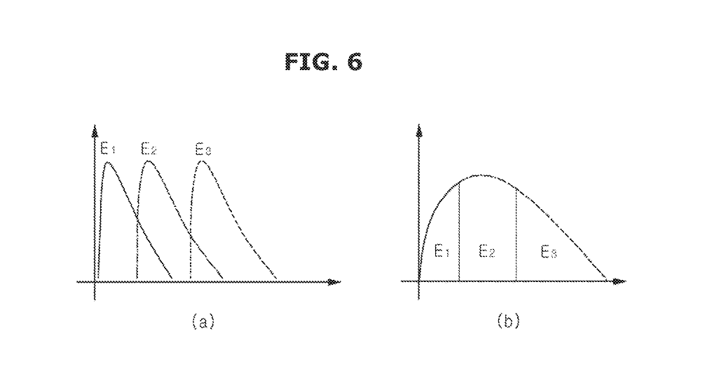

FIG. 6 is a view illustrating emitted radiation. In FIG. 6, a horizontal axis represents photon energy and a vertical axis represents intensity.

As shown in drawing (a) on the left side of FIG. 6, the radiation emission unit 100 generates a plurality of energy spectra (i.e., E1, E2, and E3) of radiation and emits the same toward the object (ob). In this case, the radiation emission unit 100 applies different tube voltages to the radiation tube 110 several times and thereby generates different respective energy bands of radiation. The radiation detection unit 200 detects the energy spectra (E1, E2, and E3) of radiation and acquires a plurality of measurement data corresponding to the respective energy spectra (E1, E2, and E3).

In addition, the radiation emission unit 100 may emit radiation having a broadband energy spectrum toward the object (ob), as shown in drawing (b) on the right side of FIG. 6. In this case, the anode 114 of the radiation emission unit 100 may be made of a material such as, for example, tungsten (W). The radiation detection unit 200 divides the broadband energy into a plurality of energy regions (i.e., E1, E2, and E3) and acquires measurement data corresponding to the respective energy regions (E1, E2, and E3).

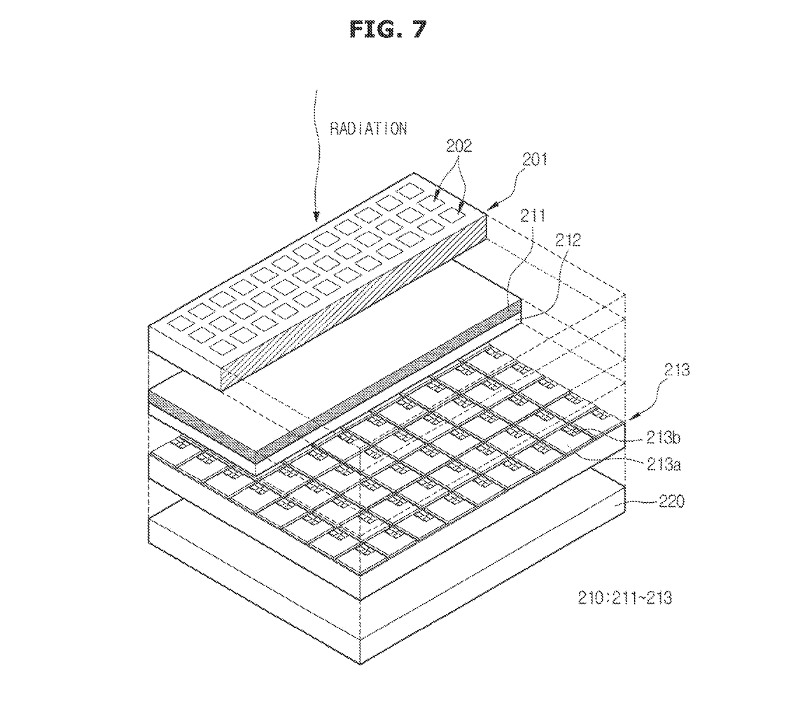

Hereinafter, an exemplary embodiment of the radiation detection unit 200 will be described in more detail. FIG. 7 is a perspective view illustrating an exemplary embodiment of the radiation detection unit.

The radiation detection unit 200 receives and detects radiation in a direct manner. Specifically, as shown in FIG. 7, the radiation detection unit 200 may include a collimator 201, a radiation detection panel 210, and a predetermined substrate 220.

The collimator 201 only transfers radiation which is emitted in a predetermined direction to the radiation detection panel 210, from among the radiation which propagates through the object (ob), and the radiation detection panel 210 collects accurate inner information which relates to the object (ob). When the radiation emitted from the radiation emission unit 100 passes through the object (ob), it may be refracted or scattered according to features, characteristics and structures of inner tissues of the object. The collimator 201 filters the radiation scattered or refracted in the object (ob), and respective pixels of the radiation detection panel 210 receive suitable radiation.

In one exemplary embodiment, the collimator 201 may include a plurality of barrier ribs made of a material which absorbs radiation, such as, for example, lead (Pb). A through hole 202 through which radiation passes may be formed between the barrier ribs. The scattered or refracted radiation is absorbed in the barrier ribs, and appropriate radiation passes through the through hole 202 and reaches the respective pixels of the radiation detection panel 210.

The radiation detection panel 210 may include a first electrode 211, a semiconductor material layer 212 mounted below the first electrode 211, and a plane plate 213 mounted below the semiconductor material layer 212. At least one second electrode (i.e., pixel electrode, 213a) may be disposed on the plane plate 213.

The first electrode 211 may have a positive (+) or negative (−) polarity and the second electrode 213a may have a negative (−) or positive (+) polarity, opposite to the polarity of the first electrode 211. A predetermined bias voltage may be applied between the first electrode 211 and the second electrode 213a.

In accordance with incidence and absorption of radiation, predetermined electric charge-hole pairs may be generated in the semiconductor material layer 212. The generated electron-hole pairs migrate to the first electrode 211 or the second electrode 213a according to the polarity of the first electrode 211 and the second electrode 213a. In one exemplary embodiment, the semiconductor material layer 212 may be a photo-conductor and, specifically, may be made of amorphous selenium.

The plane plate 213 may include at least one second electrode 213a and at least one thin film transistor 213b to which the generated charges or holes are transferred. At least one second electrode 213a may be disposed in at least one array on the plane plate 213. For example, each second electrode 213a of the plane plate 213 may be disposed in a one-dimensional array (1D array) or in a two-dimensional array (2D array), as shown in FIG. 7. The plane plate 213 may include at least one CMOS chip, according to an exemplary embodiment. One second electrode 213a and one thin film transistor 213b may be mounted on each CMOS chip.

The second electrode 213a may receive holes or negative charges transferred from the semiconductor material layer 212. The holes or negative charges transferred to the second electrode 213a may be stored in a predetermined storage device, such as, for example, a capacitor.

The thin film transistor 213b may read out an electric signal transferred from the second electrode 213a or stored in the predetermined storage device. The thin film transistor 213b is connected to each second electrode 213a and reads out the electric signal output from the second electrode 213a.

The radiation detection unit 200 receives and detects radiation in an indirect manner. In this case, a phosphor screen may be disposed between the collimator 201 and the radiation detection panel 210. The phosphor screen receives the radiation emitted from the radiation emission unit 100 and outputs predetermined light. In this case, at least one photodiode which is configured to receive the predetermined light may be disposed on the plane plate 213, instead of the second electrode 213a. The photodiode may be disposed in a one-dimensional or two-dimensional array, similarly as the second electrode 213a.

In addition, the radiation detection panel includes a scintillator which is configured to receive radiation and to output predetermined visible photons according to the received radiation, and a photo-sensor, for example a photodiode, which is configured to sense the visible photons output from the scintillator. The photodiode outputs a predetermined electric signal, such as, for example, an electric charge packet including holes or negative charges according to visible photons. The output electric charge packet may be stored in a predetermined storage device, such as, for example, a capacitor.

Further, in an exemplary embodiment, the radiation detection unit 200 may be a photon counting detector (PCD). The photon counting detector counts the number of photons having a critical energy or above, from the radiation signal and acquires predetermined measurement data. In this case, the photon counting detector amplifies the input radiation signal, compares the amplified electric signal with the critical energy, determines whether the amplified electric signal is an electric signal having an energy higher or lower than the critical energy, outputs a signal determined from comparison results, counts the number of photons having an energy higher or lower than the critical energy using the transferred signal, and outputs information obtained as a result of counting the number of photons. The photon counting information may include information relating to a radiation intensity. The photon counting detector controls the critical energy compared with the amplified electric signal and separately detects predetermined energy spectra of radiation.

The substrate 220 may be adhered to the back of the radiation detection panel 210. The substrate 200 may stably fix the radiation detection panel 210 and control readout of the electric signal detected by the radiation detection panel 210.

The radiation detection unit 200 detects the radiation which propagates through the object (ob) and acquires measurement data which relates to the inside of the object. The acquired measurement data may be transferred to the image reconstruction unit 300.

The image reconstruction unit 300 reconstructs a radiation image by using the acquired measurement data.

Specifically, the image reconstruction unit 300 generates predetermined simulation data which relates to inner tissues of the object (ob), repeatedly reduces a discrepancy between the generated simulation data and the measurement data, and thereby acquires an image reconstruction value which relates to an inner structure of the object (ob) in accordance with the acquired measurement data and produces an image by using the acquired image reconstruction value.

Specifically, the measurement data acquired by the radiation detection unit 200 may include information, such as, for example, radiation intensity, which is associated with radiation attenuated while propagating through a plurality of inner tissues of the object (ob). Information which is associated with the inner tissues of the object (ob) using the measurement data acquired by the radiation detection unit 200 is acquired. This process is referred to as “reconstruction”.

Data yᵢ which is measured at an i^{th} pixel of the radiation detection panel 210 of the radiation detection unit 200 may be given by the following Equation 2:

yᵢ=∫sᵢ(E)exp(− [symbol] ρ(x,y,z,E)dl)dE [Equation 2]

wherein yᵢ represents measurement data, and sᵢ (ε) represents an energy spectrum of radiation emitted from the radiation emission unit 100.

Further, the function ρ exhibits attenuation properties at a position (x,y,z) of the object when radiation of the energy spectrum E is emitted.

Furthermore, [symbol] ᵢ(•)dl, which is applied to the function ρ, represents a line projection of the function ρ, and specifically a line projection of attenuation properties of substances through which radiation propagates. In particular, [symbol] ᵢ(•)dl is a parameter which reflects attenuation properties of substances present between the radiation emission unit 100 and the i^{th} pixel of the radiation detection panel 210 of the radiation detection unit 200. Because plural inner substances of the object (ob) attenuate radiation which reaches the substances according to inherent properties thereof, radiation reaching the radiation detection panel 210 is attenuated by the substances. Accordingly, the line projection reflects attenuation properties of inner substances of the object (ob).

In a case in which the measurement data is given as expressed in Equation 2, since the measurement data yᵢ is already acquired, acquisition of a value of ρ(x,y,z,E) using the acquired measurement data yᵢ is required in order to find inner substances of the object. Acquisition of the value of ρ(x,y,z,E) by using the acquired measurement data yᵢ represents a reconstruction which may be performed by the radiation imaging apparatus.

Hereinafter, ρ(x,y,z,E) will be referred to as an image reconstruction value.

The image reconstruction unit 300 acquires predetermined simulation data (ŷᵢ) and then acquires an image reconstruction value, that is, ρ(x,y,z,E), an approximate value of the image reconstruction value, and/or an assumed value of the image reconstruction value by using the simulation data, thereby reconstructing an image.

Specifically, after producing the simulation data, the image reconstruction unit 300 acquires a correction value which reduces and/or minimizes a discrepancy between the simulation data and the measurement data in order to determine and acquire an image reconstruction value, and reconstructs an image by using the determined and acquired image reconstruction value. In particular, the image reconstruction unit 300 determines and acquires the image reconstruction value corresponding to the simulation data when a discrepancy between the simulation data and the measurement data is reduced and/or minimized, and reconstructs an image by using the determined and acquired image reconstruction value.

As the discrepancy between the simulation data acquired by the image reconstruction unit 300 and the measurement data detected by the radiation detection unit 200 decreases, the simulation data and the measurement data may be similar to each other. If the discrepancy between the simulation data and the measurement data is equal to zero or an extremely small value, the simulation data and the measurement data may be identical or considerably approximate. In a case in which the simulation data and the measurement data are identical or considerably approximate, when the image reconstruction value corresponding to the simulation data which is identical or considerably approximate to the measurement data is acquired, the image reconstruction value is acquired according to the actual measurement data. Accordingly, a radiation image, which is identical or similar to inner substances of the object (ob) according to the actual measurement data, is reconstructed.

The image reconstruction unit 300 acquires an image reconstruction value corresponding to the simulation data by using a predetermined function (hereinafter, referred to as a “discrepancy function”) of the discrepancy between the simulation data and the measurement data as described above. The simulation data may be identical or similar to the measurement data. Specifically, the image reconstruction unit 300 acquires simulation data corresponding to a minimal value of the predetermined discrepancy function and thereby acquires the image reconstruction value corresponding to the simulation data which is identical or similar to the measurement data.

Further, in an exemplary embodiment, the image reconstruction unit 300 acquires an image reconstruction value by using a predetermined cost function and simulation data. In this case, the image reconstruction unit 300 acquires the image reconstruction value by using the predetermined cost function associated with the discrepancy function, instead of the discrepancy function. Specifically, the image reconstruction unit 300 acquires new simulation data of the minimal value of the cost function, instead of the minimal value of the discrepancy function, and thereby acquires an image reconstruction value corresponding to the simulation data identical or similar to the measurement data.

When the image reconstruction unit 300 uses a cost function, it repeatedly acquires an image reconstruction value of the cost function and thereby acquires a final image reconstruction value used for image reconstruction. Specifically, the image reconstruction unit acquires new simulation data by using the simulation data and the predetermined cost function, and acquires new simulation data or an image reconstruction value by using the acquired new simulation data and the predetermined cost function. In this case, the cost functions used for respective operations may be identical or different. In addition, the cost function used for respective operations may be determined by the simulation data. By repeating these processes, the image reconstruction unit 300 acquires a minimal value of the cost function which is identical or approximate to a minimal value of the discrepancy function, and acquires an image reconstruction value corresponding to the minimal value of the cost function, which will be used for image reconstruction.

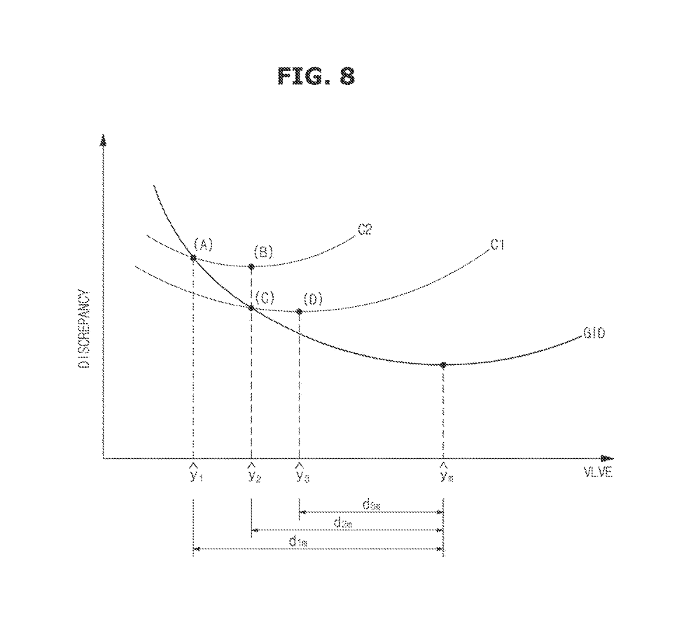

FIG. 8 is a graph illustrating an operation of the image reconstruction unit.

In FIG. 8, a horizontal axis represents simulation data or measurement data and a vertical axis represents a discrepancy between simulation data and measurement data. In FIG. 8, GID represents a discrepancy function, indicating a discrepancy between simulation data or measurement data, and c1 and c2 represent cost functions.

As described above, the image reconstruction unit acquires an image reconstruction value corresponding to the simulation data ( [symbol] of FIG. 8) when the discrepancy function is a minimal value and thereby reconstructs an image. When it is difficult to use a discrepancy function, such as, for example, when it is difficult to directly calculate the discrepancy function, resources of apparatuses required for direct calculation of the discrepancy function are large, or time required for discrepancy function calculation is long, a predetermined cost function corresponding to the discrepancy function may be used.

For example, in FIG. 8, the first cost function (c1) and the second cost function (c2) are used. Hereinafter, a process for acquiring an image reconstruction value using a cost function will be described with reference to FIG. 8.

First, first simulation data ( [symbol] ) is acquired.

In this case, as shown in FIG. 8, there may be a discrepancy of about d₁ₘ between the first simulation data ( [symbol] ) and the simulation data ( [symbol] ) with respect to minimizing the discrepancy function. This discrepancy may be representative of an error between an actual inner substance of the object (ob) and an image to be reconstructed. When the error between the actual inner substance of the object (ob) and the image to be reconstructed is unacceptable, the second simulation data ( [symbol] ) is acquired.

When the error between the actual inner substance of the object (ob) and the reconstructed image is unacceptable, the second cost function (c2) corresponding to the discrepancy function (GID) along the first simulation data ( [symbol] ) is acquired. The second cost function (c2) may meet the discrepancy function (GID) at a value corresponding to the first simulation data ( [symbol] ) (i.e., Point A).

In addition, new simulation data, that is, second simulation data ( [symbol] ) minimizing the second cost function (c2) is acquired (i.e., Point B). There may be a discrepancy of about d₂ₘ between the newly acquired second simulation data ( [symbol] ) and the simulation data ( [symbol] ) with respect to minimizing the discrepancy function, as shown in FIG. 8. When this discrepancy, that is, the error between the actual inner substance of the object (ob) and the image, is unacceptable, the third simulation data ( [symbol] ) is acquired.

In this case, the first cost function (c1) corresponding to the discrepancy function (GID) along the second simulation data ( [symbol] ) is acquired. The first cost function (c1) may meet the discrepancy function (GID) at a value corresponding to the second simulation data ( [symbol] ) (i.e., Point C).

New simulation data minimizing the first cost function (c1), that is, the third simulation data ( [symbol] ), is acquired (i.e., Point D). There may be a discrepancy of about d₃ₘ between the newly acquired third simulation data ( [symbol] ) and the simulation data ( [symbol] ) minimizing the discrepancy function, as shown in FIG. 8. When this discrepancy is unacceptable, a new cost function along the third simulation data ( [symbol] ) is acquired and new simulation data is thus acquired.

By repeating such a process, a minimal value of the cost function, which is identical or approximate to the minimal value of the discrepancy function (GID), is acquired, and an image reconstruction value corresponding to the simulation data of the acquired minimal value of the cost function is thus acquired.

As described above, the image reconstruction unit 300 repeatedly calculates respective simulation data, i.e., ( [symbol] ) to ( [symbol] ), and thereby acquires an image reconstruction value. In addition, the image reconstruction unit 300 acquires an image reconstruction value by using a predetermined Equation acquired according to previously calculated results, without repeatedly calculating the respective simulation data ( [symbol] ) to ( [symbol] ).

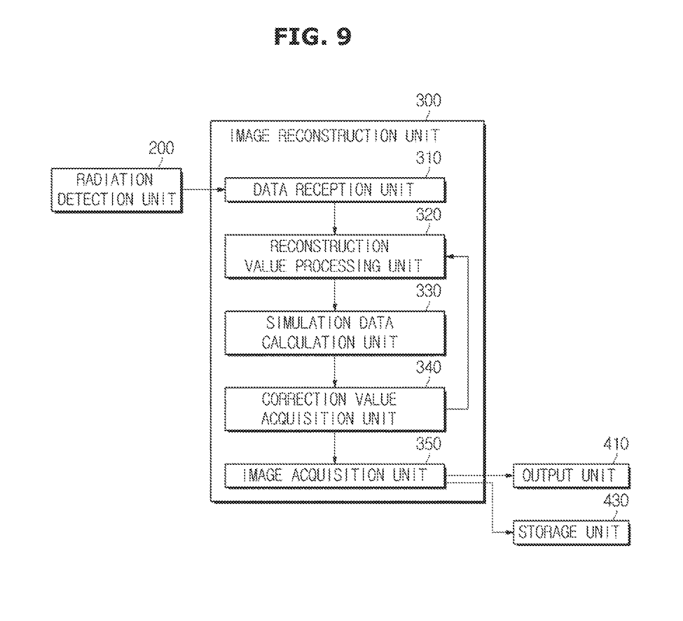

FIG. 9 is a block diagram illustrating an exemplary embodiment of the image reconstruction unit.

As shown in FIG. 9, in an exemplary embodiment, the image reconstruction unit 300 may include a data reception unit (also referred to herein as a data receiver) 310, a reconstruction value processing unit (also referred to herein as a reconstruction value processor) 320, a simulation data calculation unit (also referred to herein as a simulation data calculator) 330, a correction value acquisition unit (also referred to herein as a correction value generator) 340 and an image acquisition unit (also referred to herein as an image generator) 350.

The data reception unit 310 may receive measurement data transferred from the radiation detection unit 200. The measurement data received by the data reception unit 310 may include raw data which is directly transferred from the radiation detection unit 200, or data which is obtained by amplification or predetermined conversion, for example, analog to digital conversion, of the raw data.

The reconstruction value processing unit 320 initializes the image reconstruction value and/or updates the image reconstruction value according to a correction value acquired by the correction value acquisition unit 340. In this case, the image reconstruction value may include information which is associated with (K+α) inner substances of the object.

The reconstruction value processing unit 320 sets the image reconstruction value to a predetermined initial value and thereby initializes the image reconstruction value. In this case, the predetermined initial value may be randomly assigned by a user or a system designer. The predetermined initial value may relate to and/or be determined by any one or more of a type of the radiation imaging apparatus, a type of the object (ob), an area of the object (ob) to be imaged, a type of contrast agent, a type of energy spectrum of emitted radiation, a type of radiation image to be acquired, and/or the like.

The simulation data calculation unit 330 calculates predetermined simulation data by using the initialized image reconstruction value and/or the updated image reconstruction value. In this case, the simulation data calculation unit 330 calculates the simulation data according to the received measurement data. The simulation data is necessarily not calculated. According to an exemplary embodiment, the simulation data may be separately not calculated.

The correction value acquisition unit 340 acquires a correction value of the image reconstruction value by using the image reconstruction value of the reconstruction value processing unit 320 and/or the simulation data calculated by the simulation data calculation unit 330. In this case, the correction value reduces and/or minimizes a discrepancy between the measurement data and the simulation data. In particular, the correction value corresponds to a simulation data minimizing discrepancy function (GID) and/or cost functions (c1, c2).

The correction value acquired by the correction value acquisition unit 340 may be transferred to the image acquisition unit 350 and/or to the reconstruction value processing unit 320.

The image acquisition unit 350 reconstructs an image, based on the correction value acquired by the correction value acquisition unit 340, and thereby acquires a radiation image. In another exemplary embodiment, the image acquisition unit 350 reconstructs the image by using the image reconstruction value corresponding to the simulation data initialized by the reconstruction value processing unit 320.

The radiation image acquired by the image acquisition unit 350 may be transferred to an output unit 410, such as, for example, a display device, and be displayed to a user.

If necessary, the radiation image acquired by the image acquisition unit 350 may be simultaneously or separately transferred to a separate storage unit 430, when it is transferred to the output unit 410. In this case, the radiation image may be transferred to the separate storage unit 430 before being transferred to the output unit 410. The storage unit 430 may temporarily or permanently store the transferred radiation image. The storage unit 430 may include an interior memory device mounted in the radiation detection unit 200 or the console 10, and/or an exterior memory device which is connectable to the console 10 or the like by using a wired or wireless data network. In addition, the storage unit 430 may include a magnetic memory device which uses a magnetic disc and/or a memory device which uses a predetermined semiconductor chip. In addition, any one or more of a variety of storage media to store data may be used as the storage unit 430.

Further, the correction value acquired by the correction value acquisition unit 340 may be transferred to the reconstruction value processing unit 320 and the reconstruction value processing unit 320 may update the image reconstruction value by using the transferred correction value. In this case, the reconstruction value processing unit 320 may discard the previously set image reconstruction value. The newly updated image reconstruction value may be transferred to the simulation data calculation unit 330.

When the correction value acquired by the correction value acquisition unit 340 is transferred to both the reconstruction value processing unit 320 and the image acquisition unit 350, the image reconstruction value updated by the reconstruction value processing unit 320 will be the same as the image reconstruction value used for image reconstruction, that is, the correction value.

According to an exemplary embodiment, the image reconstruction value is first updated by using the correction value acquired by the correction value acquisition unit 340, the updated image reconstruction value is then transferred to the image acquisition unit 350, and image reconstruction is carried out by the image acquisition unit 350.

Hereinafter, an exemplary embodiment of an operation of the image reconstruction unit 300 will be described in more detail with reference to an Equation.

A j^{th} voxel of an object to be reconstructed is expressible in accordance with the following Equation 3:

ρⱼ=[ρⱼ^{B}^{₀}, . . . ,ρⱼ^{B}^{_{α}},ρⱼ^{C}^{₁}, . . . ,ρⱼ^{C}^{_{Nc}}]^{T}εR^{(K+α)×1} [Equation 3]

wherein N_{c} represents the number of contrast agents in which N_{c}=K−1, and ρⱼ^{B} represents a basis density of a substance to be virtually separated wherein ρⱼ is used as the image reconstruction value.

A vector of a relative partial density of the j^{th} voxel of the object to be reconstructed is expressible in accordance with the following Equation 4:

xⱼ=[xⱼ^{S}^{₀}, . . . ,xⱼ^{S}^{_{α}},xⱼ^{C}^{₁}, . . . ,xⱼ^{C}^{_{Nc}}]^{T}εR^{(K+α)×1} [Equation 4]

wherein α+1 represents the number of substances to be virtually separated.

A relation between ρⱼ and xⱼ is expressible in accordance with the following Equation 5:

$$\begin{matrix}{x}_{j} = [ \begin{matrix}f ( {ρ}_{j}^{B} ) & {0}_{( α + 1 ) \times Nc} \\ {0}_{Nc \times 1} & {I}_{Nc \times Nc}\end{matrix} ] [ \begin{matrix}{ρ}_{j}^{B} \\ {ρ}_{j}^{{c}_{1}} \\ ⋮ \\ {ρ}_{j}^{{c}_{Nc}}\end{matrix} ] \\ = {[ \begin{matrix}{ρ}_{j}^{B} {f ( {ρ}_{j}^{B} )}^{T} & 0 \\ 0 & {ρ}_{j}^{{c}_{1}} , … , {ρ}_{j}^{{c}_{Nc}}\end{matrix} ]}^{T}\end{matrix} \qquad [ Equation 5 ]$$

wherein f(ρⱼ^{B}) is a function which defines respective fractions of (α+1) substances from the basis density and is expressible in accordance with the following Equation 6:

f(ρⱼ^{B})=[f^{B}^{₀}(ρⱼ^{B}), . . . ,f^{B}^{_{α}}(ρⱼ^{B})]^{T}εR^{(α+1)×1} [Equation 6]

A line projection of (K+α) is expressible in accordance with the following Equations 7, 8, and 9:

$$\begin{matrix}{p}_{i} = {[ {p}_{i}^{{B}_{0}} … , {p}_{i}^{{B}_{a}} , {p}_{i}^{{C}_{1}} , … , {p}_{i}^{{C}_{Nc}} ]}^{T} & [ Equation 7 ] \\ {p}_{i}^{{s}_{k}} = {\sum }_{j} {a}_{ij} {f}^{{s}_{k}} ( {p}_{i}^{B} ) \cdot {p}_{i}^{B} & [ Equation 8 ] \\ {p}_{i}^{{C}_{Nk}} = {\sum }_{j} {a}_{ij} {p}_{i}^{{C}_{Nk}} & [ Equation 9 ]\end{matrix}$$

In Equations 8 and 9, aᵢⱼ represents a level at which a j^{th} voxel value of the object (ob) contributes to an i^{tb} pixel of the radiation detection panel 210. In particular, aᵢⱼ represents a level at which the j^{th} voxel value contributes to measurement data.

In addition, a mass attenuation vector of (K+α) substances is expressible in accordance with the following Equation 10:

$$( \frac{μ}{ρ} ) ( E ) = {[ \frac{{μ}^{( 1 )} ( E )}{{ρ}^{( 1 )}} , … , \frac{{μ}^{( K + α )} ( E )}{{ρ}^{( K + α )}} ]}^{T} ∈ {R}^{( K + α ) \times 1} \qquad [ Equation 10 ]$$

As expressed in Equations 3 to 10 above, data yᵢ measured at the i^{th} pixel of the radiation detection panel 210 may be expressible in accordance with the following Equation 11. In particular, Equation 2 may be rewritten as the following Equation 11:

$${y}_{i} = {\sum }_{E} {s}_{i} ( E ) exp ( - {( \frac{μ}{β} )}^{T} ( E ) {p}_{i} ) \qquad [ Equation 11 ]$$

Under the assumption that a model of measurement data yᵢ is determined as expressed in Equation 11 above, a correction value of the image reconstruction value may be acquired by acquiring a value which reduces and/or minimizes a predetermined discrepancy function which is associated with measurement data yᵢ and simulation data [symbol] .

Specifically, the predetermined discrepancy function is expressible in accordance with the following Equation 12:

$$G ( f ( r ) \vert \vert g ( r ) ) = \int f ( r ) ψ ( \frac{f ( r )}{g ( r )} ) d r \qquad [ Equation 12 ]$$

wherein ψ(x) is a convex function which reaches a minimal value when x is equal to 1. In particular, ψ(x) satisfies the following Equation 13.

$$\begin{matrix}argmin \\ v\end{matrix} ψ ( v ) = 1 \qquad [ Equation 13 ]$$

Specifically, ψ(x) reaches a minimal value when two functions, that is, f(r) and g(r), are identical. Accordingly, in accordance with Equations 12 and 13, a discrepancy between two functions, that is, f(r) and g(r), may be defined. Accordingly, when a model of measurement data yᵢ and a model of simulation data ( [symbol] ) are applied to the respective functions, the discrepancy between the measurement data yᵢ and the simulation data ( [symbol] ) is defined and as a result, a value which reduces and/or minimizes the discrepancy between the measurement data yᵢ and the simulation data ( [symbol] ) is calculated. When a value of an inner substance corresponding to the value which reduces and/or minimizes the discrepancy between the measurement data yᵢ and the simulation data ( [symbol] ), for example, a density ρⱼ of the inner substance of the object (ob), is acquired, a reconstruction value of the inner substance of the object (ob) is acquired. As the reconstruction value becomes more accurate, measurement data yᵢ and simulation data ( [symbol] ) may be identical or considerably approximate.

In this case, a reconstruction value of the inner substance corresponding to the value which reduces and/or minimizes the discrepancy between the measurement data yᵢ and the simulation data ( [symbol] ) may be acquired by any one or more of a variety of methods in accordance with the expression of the discrepancy function depicted by Equation 12.

In an exemplary embodiment, the discrepancy function depicted by Equation 12 may be expressible in accordance with the following Equation 14:

$${\sum }_{i} {G}_{R} ( {y}_{i} \vert \vert ) = {\sum }_{i} {v}_{i} \cdot ψ ( \frac{{y}_{i}}{\hat{{y}_{i}}} ) \qquad [ Equation 14 ]$$

wherein yᵢ represents measurement data and ( [symbol] ) represents simulation data.

The simulation data ( [symbol] ) may be acquired by the simulation data calculation unit 330 of the image reconstruction unit 300.

The simulation data calculation unit 330 calculates simulation data ( [symbol] ) of the inner substance of the object (ob) by using Equation 11 above. When the reconstruction value processing unit 320 initializes the image reconstruction value and thereby sets an initial value of the image reconstruction value, the simulation data calculation unit 330 applies the initial value of the image reconstruction value to Equation 11 above and thereby calculates simulation data ( [symbol] ) of the inner substance of the object (ob).

Further, an inequality which is represented by the following Equation 15 is obtained by developing Equation 14.

$${\sum }_{i} G ( {y}_{i} \vert \vert ) \leq {\sum }_{i , E} {d}_{i} ( {p}_{i} , E ) \cdot ψ ( \frac{{d}_{i} ( {p}_{i} , E )}{{m}_{i} ( {p}_{i} , E )} ) \qquad [ Equation 15 ]$$

wherein dᵢ(pᵢ,E) and mᵢ(pᵢ,E) are expressible in accordance with the following Equations 16 and 17.

$$\begin{matrix}{d}_{i} ( {p}_{i} , E ) = {m}_{i} ( {p}_{i} , E ) \cdot ( \frac{{y}_{i}}{} ) & [ Equation 16 ] \\ {m}_{i} ( {p}_{i} , E ) = {s}_{i} ( E ) exp ( - {( \frac{μ}{ρ} )}^{T} ( E ) {p}_{i} ) & [ Equation 17 ]\end{matrix}$$

In Equation 15, when the right side is assumed as a surrogate objective function c, the surrogate objective function c is convex with respect to a specific ψ function. For example, the specific function ψ is expressible in accordance with the following Equations 18 and 19:

$$\begin{matrix}{ψ}_{1} ( x ) = {x}^{α} + ( \frac{α}{β} ) {x}^{- β} & [ Equation 18 ] \\ {ψ}_{2} ( x ) = log ( x ) + \frac{1}{β} {x}^{- β} & [ Equation 19 ]\end{matrix}$$

When the specific function ψ is given by Equation 18, the surrogate objective function c is convex, if α is greater than zero (with a proviso that α is not equal to zero) and β is equal to or greater than zero. When the specific function ψ is given by Equation 19, the surrogate objective function c is convex if β is greater than zero (with the proviso that β is not equal to zero).

Equation 17 is more simply developed as shown in the following Equation 20 because Equation 17 is convex.

$${q}_{i} ( {p}_{i} , E ; ) = {c}_{i} ( , E ) + {{g}_{i} ( , E )}^{T} ( {p}_{i} - ) + \frac{1}{2} {( {p}_{i} - )}^{T} {H}_{i} ( , E ) ( {p}_{i} - ) \qquad [ Equation 20 ]$$

Equation 20 may be represented more simply by the following Equation 21, if an obtained marginal sum of energy is represented by superscript ξ.

$${q}_{i}^{ξ} ( {p}_{i} ; ) = {{g}_{i}^{ξ} ( )}^{T} ( {p}_{i} - ) + \frac{1}{2} {( {p}_{i} - )}^{T} {H}_{i}^{ξ} ( ) ( {p}_{i} - ) \qquad [ Equation 21 ]$$

The following Equation 22 is defined:

$${χ}_{i , j}^{ξ} ( {p}_{i} ; ) = {α}_{ij} {q}_{i}^{ξ} ( \frac{{a}_{ij}}{{α}_{ij}} ( {x}_{j} - \hat{{x}_{j}} ) + ; ) \qquad [ Equation 22 ]$$

wherein αᵢⱼ is expressible in accordance with the following Equations 23 and 24:

$$\begin{matrix}{α}_{ij} = \frac{{a}_{ij}}{{γ}_{i}} & [ Equation 23 ] \\ {γ}_{i} = {\sum }_{j} {a}_{ij} & [ Equation 24 ]\end{matrix}$$

As a result, a relation between Equation 21 and Equation 22 may be expressible in accordance with the following Equation 25:

$${\sum }_{i} {q}_{i}^{ξ} ( {p}_{i} ; ) \leq {\sum }_{i , j} {χ}_{i , j}^{ξ} ( {p}_{i} ; ) \qquad [ Equation 25 ]$$

Equations 20, 21, and 22 above may be used for the cost function described above.

An update equation represented by the following Equation 26 is obtained when Newton's method is applied to Equation 22 above.

$${ρ}_{j} = - {( {( \frac{\partial {x}_{j}}{\partial {ρ}_{j}} )}^{T} ( {\sum }_{i} {a}_{ij} {γ}_{i} {H}_{i}^{ɛ} ( ) ) ( \frac{\partial {x}_{j}}{\partial {ρ}_{j}} ) )}^{- 1} ( {( \frac{\partial {x}_{j}}{\partial {ρ}_{j}} )}^{T} ( {\sum }_{i} {a}_{ij} {g}_{i}^{ɛ} ( ) ) \qquad [ Equation 26 ]$$

wherein ρⱼ represents a correction value of a newly acquired image reconstruction value and [symbol] represents an image reconstruction value corresponding to simulation data.

According to an exemplary embodiment, [symbol] may be an initialized image reconstruction value. In particular, in a case in which an image is reconstructed, based on the measurement data, when the image reconstruction value is initialized, the correction value of the image reconstruction value, that is, ρⱼ, is acquired by using the initialized image reconstruction value [symbol] , measurement data, and simulation data.

Furthermore, when the correction value of the image reconstruction value is applied to [symbol] again and Equation 26 is calculated, a correction value ρⱼ for correcting the image reconstruction value again is acquired.

By repeating such a process, an appropriate image reconstruction value may be acquired.

Further, in Equation 26,

$$\frac{\partial x i}{\partial ρ j}$$

is expressible in accordance with the following Equation 27:

$$\frac{\partial {x}_{j}}{\partial {ρ}_{j}} = [ \begin{matrix}{ρ}_{j}^{B} \cdot \frac{\partial f ( {ρ}_{j}^{B} )}{\partial {ρ}_{j}^{B}} + f ( {ρ}_{j}^{B} ) & 0 \\ 0 & {I}_{N c}\end{matrix} ] ∈ {R}^{( K \vert 1 ) < K} . \qquad [ Equation 27 ]$$

Still further, Hᵢ^{ε} may be expressible in accordance with the following Equation 28 and gⱼ^{e} may be expressible in accordance with the following Equation 30:

$${H}_{i}^{ɛ} ( ) = ( {e}_{i}^{3} {φ}^{″} ( {e}_{i} ) + {e}_{i}^{2} {φ}^{′} ( {e}_{i} ) ) {\sum }_{E} ( E ) M ( E ) \qquad [ Equation 28 ]$$

wherein M(E) is expressible in accordance with the following Equation 29:

$$\begin{matrix}M ( E ) = ( \frac{μ}{ρ} ) \cdot {( \frac{μ}{ρ} )}^{T} & [ Equation 29 ] \\ {g}_{i}^{ɛ} ( ) = {e}_{i}^{2} {φ}^{′} ( {e}_{i} ) {\sum }_{E} ( E ) ( \frac{μ}{ρ} ) ( E ) & [ Equation 30 ]\end{matrix}$$

wherein eᵢ is expressible in accordance with the following Equation 31:

$${e}_{i} = \frac{{y}_{i}}{} \qquad [ Equation 31 ]$$

At least one calculation of Equations 26, 27, 28, 29, 30, and 31 described above may be performed by the correction value acquisition unit 340. When the correction value of the image reconstruction value, that is, ρⱼ, is acquired, the acquired correction value ρⱼ is transferred to the reconstruction value processing unit 320 and is used to update the image reconstruction value, and/or is transferred to the image acquisition unit 350 and is used for image reconstruction.

In a case in which the image reconstruction value is updated by using the correction value ρⱼ, the correction value ρⱼ is used as [symbol] in Equation 26 above for repeated correction value calculation.

Through repetition of such a process, the correction value ρⱼ is identical or considerably similar to the original density of the substance of the object (ob). Accordingly, the reconstructed image is identical or considerably similar to the actual structure of the object (ob).

Equation 26 is expressible in a simplified form in accordance with the following Equation 32:

ρⱼ= [symbol] −Nⱼ⁻¹dⱼ [Equation 32]

In this case, a first adjustment value Nⱼ and a second adjustment value dⱼ are expressible in accordance with the following Equations 33 and 34. The first adjustment value Nⱼ and the second adjustment value dⱼ are induced by the generalized information theoretic discrepancy function and/or an approximation function of the information theoretic discrepancy, as described above.

$$\begin{matrix}{N}_{j} = ( {( \frac{\partial {x}_{j}}{\partial {ρ}_{j}} )}^{T} ( {\sum }_{i} {a}_{ij} {γ}_{i} {H}_{i}^{ɛ} ( ) ) ( \frac{\partial {x}_{j}}{\partial {ρ}_{j}} ) ) & [ Equation 33 ] \\ {d}_{j} = ( {( \frac{\partial {x}_{j}}{\partial {ρ}_{j}} )}^{T} ( {\sum }_{i} {a}_{ij} {g}_{i}^{ɛ} ( ) ) & [ Equation 34 ]\end{matrix}$$

In another exemplary embodiment, the discrepancy function represented by Equation 12 is expressible in accordance with the following Equation 35:

$${\sum }_{i} {G}_{N} ( {z}_{i} ‖ ) = {\sum }_{i} ( z + i + {E}_{i} + {B}_{i} ) \cdot ψ ( \frac{{z}_{i} + {E}_{i}}{+ {E}_{i}} ) \qquad [ Equation 35 ]$$

wherein ψ function is a positive (+) function which has positive values with respect to all variables and Bᵢ is a constant. Bᵢ has an effect of correcting an error generated by addition of a weight to the background of a radiation image by adding a predetermined penalty to a vacancy.

Eᵢ is a constant which makes \hat{z}+Eᵢ positive at all times.

Bᵢ and Eᵢ are expressible in accordance with the following Equations 36 and 37 using a feature that measurement data yᵢ is equal to 1 or a value which is approximately equal to 1 in the background and is less than 1 in other parts.

Bᵢ=ω_{B,i}·yᵢ^{α}^{_{B,i}} [Equation 36]

Eᵢ=ω_{E,i}·yᵢ^{α}^{_{B,i}} [Equation 37]

Further, zᵢ is expressible in accordance with the following Equation 38.

$${z}_{i} = - log {y}_{i} = - log ( {\sum }_{E} {s}_{i} ( E ) exp ( - {( \frac{μ}{ρ} )}^{T} ( E ) {p}_{i} ) ) \qquad [ Equation 38 ]$$

In particular, zᵢ may be obtained by further adding a negative logarithmic value to the model of the measurement data yᵢ.

An equation which is expressible in accordance with the following Equation 39 is induced by using Equation 35.

$${\sum }_{i} G ( {z}_{i} ‖ ) \leq {\sum }_{i , E} ( {β}_{i} ( E ) + {t}_{i} ( {p}_{i} , E ) \cdot ( \frac{{z}_{i} + {E}_{i}}{+ {E}_{i}} ) \qquad [ Equation 39 ]$$

wherein βᵢ is a positive function, a marginal sum of which is Bᵢ of Equation 35. In addition, tᵢ (pᵢ, E) is expressible in accordance with the following Equation 40:

tᵢ(pᵢ,E)=sᵢ(E)μ(E)^{T}pᵢ+εᵢ(E) [Equation 40]

wherein εᵢ(E) is a positive function, a sum of which satisfies Eᵢ.

The following Equation 41 may be derived from Equation 39 and as a result, a cost function represented by Equation 41 may be acquired.

$${\sum }_{i} G ( {z}_{i} ‖ ) \leq {\sum }_{i , E} ( {β}_{i} ( E ) + {f}_{i} ( {p}_{i} , E ) ) \cdot ψ ( \frac{{f}_{i} ( {p}_{i} , E )}{{t}_{i} ( {p}_{i} , E )} ) \qquad [ Equation 41 ]$$

wherein fᵢ (pᵢ, E) is determined as expressible in accordance with the following Equation 42.

$$\begin{matrix} {f}_{i} ( {p}_{i} , E ) = {t}_{i} ( {p}_{i} , E ) \cdot ( \frac{{z}_{i} + {E}_{i}}{+ {E}_{i}} ) & [ Equation 42 ] \\ {\sum }_{i , E} {c}_{i} ( {p}_{i} , E ; ) = {\sum }_{i , E} ( {β}_{i} ( E ) + ( E ) ) \cdot ψ ( \frac{ ( , E )}{{t}_{i} ( {p}_{i} , E )} ) & [ Equation 43 ]\end{matrix}$$

Equation 41 is similar to Equation 15 above. Accordingly, an update equation may be obtained in the same manner as in Equations 15 to 25.

In this case, the update equation is expressible in accordance with the following Equation 44.

$${ρ}_{j} = - {( {( \frac{\partial {x}_{j}}{\partial {ρ}_{j}} )}^{T} ( {\sum }_{i} {a}_{ij} {γ}_{i} {H}_{i}^{ɛ} ( ) ) ( \frac{\partial {x}_{j}}{\partial {ρ}_{j}} ) )}^{- 1} ( {( \frac{\partial {x}_{j}}{\partial {ρ}_{j}} )}^{T} ( {\sum }_{i} {a}_{ij} {g}_{i}^{ɛ} ( ) ) \qquad [ Equation 44 ]$$

Comparing Equation 26 with Equation 44, update equations are identical. Hᵢ^{ε} and gⱼ^{ε} of Equation 44 are different from Hᵢ^{ε} and gⱼ^{ε} of Equation 26.

In Equation 44, Hᵢ^{ε} is expressible in accordance with the following Equation 45 and gⱼ^{ε} is expressible in accordance with the following Equation 46.

$$\begin{matrix}{H}_{i}^{ɛ} ( ) = ( 2 {e}_{i} {φ}^{′} ( {e}_{i} ) + {e}_{i}^{2} {φ}^{″} ( {e}_{i} ) ) \times {\sum }_{E} \frac{{s}_{i}^{2} ( E ) M ( E )}{{t}_{i} ( E )} ( {e}_{i} + \frac{{β}_{i} ( E )}{{t}_{i} ( E )} ) & [ Equation 45 ] \\ {g}_{i}^{ɛ} ( ) = - {e}_{i} {φ}^{′} ( {e}_{i} ) {\sum }_{E} {s}_{i} ( E ) ( \frac{μ}{ρ} ) ( E ) ( {e}_{i} + \frac{{β}_{i} ( E )}{{t}_{i} ( E )} ) & [ Equation 46 ]\end{matrix}$$

wherein eᵢ is expressible in accordance with the following Equation 47:

$${e}_{i} = \frac{{z}_{i} + {E}_{i}}{+ {E}_{i}} \qquad [ Equation 47 ]$$

In another exemplary embodiment, the discrepancy function represented by Equation 12 is expressible in accordance with the following Equation 48:

$${\sum }_{i} {G}_{U} ( {z}_{i} \vert \vert ) = {\sum }_{i} ( 1 - \frac{{z}_{i} + {E}_{i}}{Y} ) \cdot ψ ( \frac{{z}_{i} + {E}_{i}}{+ {E}_{i}} ) \qquad [ Equation 48 ]$$

wherein zᵢ is expressible in accordance with Equation 38 above. In addition, Y is a positive constant. Further, in this case, it is assumed that the ψ (x) function has a negative value, when x has a value which is greater than zero and lower than a predetermined positive value.

Update equations, which are the same as Equations 26 and 44, may be acquired via calculation of a method of the exemplary embodiment described above using Equation 48. Like Equation 44, Hᵢ^{ε} and gⱼ^{ε} of Equation 48 may be different from Hᵢ^{ε} and gⱼ^{ε} of Equations 26 and 44. In Equation 48, Hᵢ^{ε} is expressible in accordance with the following Equation 49 and gⱼ^{ε} is expressible in accordance with the following Equation 50.

$$\begin{matrix}{H}_{i}^{ɛ} ( ) = \frac{1}{Y} ( 2 {e}_{i} {φ}^{′} ( {e}_{i} ) + {e}_{i}^{2} {φ}^{″} ( {e}_{i} ) ) \times {\sum }_{E} \frac{{s}_{i}^{2} ( E ) M ( E )}{{t}_{i} ( E )} ( \frac{U ( E )}{{t}_{i} ( E )} - {e}_{i} ) & [ Equation 49 ] \\ {g}_{i}^{ɛ} ( ) = \frac{1}{Y} {e}_{i} {φ}^{′} ( {e}_{i} ) {\sum }_{E} {s}_{i} ( E ) ( \frac{μ}{ρ} ) ( E ) ( {e}_{i} - \frac{U ( E )}{{t}_{i} ( E )} ) & [ Equation 50 ]\end{matrix}$$

The value of eᵢ is the same as in Equation 47. Further, U(E) is a function which has the same energy sum as Y.

Hereinafter, exemplary embodiments of an image reconstruction method will be described.

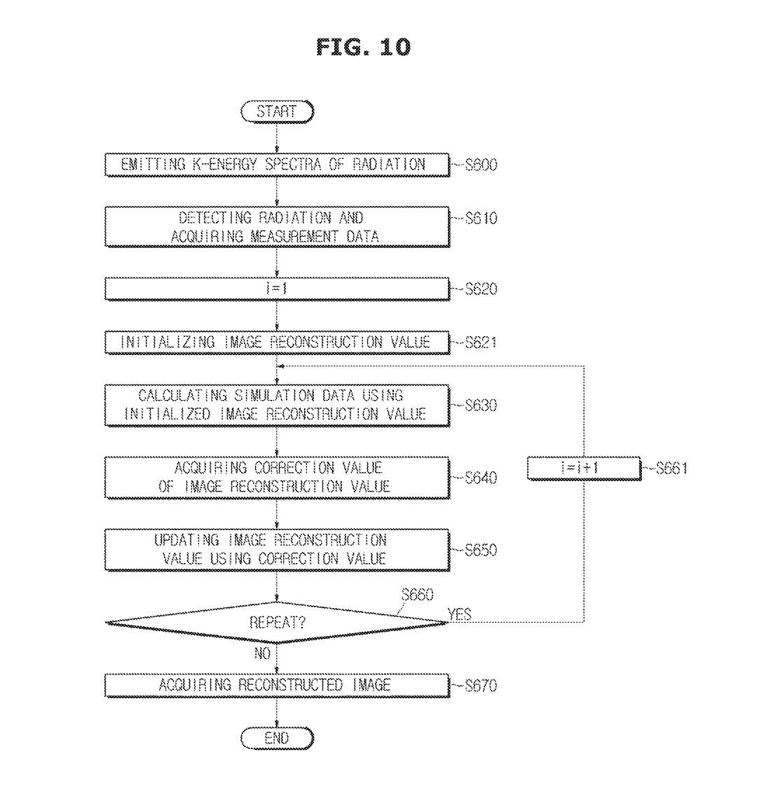

FIG. 10 is a flowchart illustrating an image reconstruction method which is executable by using a radiation imaging apparatus, according to an exemplary embodiment.

The radiation imaging apparatus may be a polychromatic radiation imaging apparatus which emits polychromatic radiation toward an object (ob). In addition, the radiation imaging apparatus may be a computed tomography (CT) apparatus.

As shown in FIG. 10, in accordance with an exemplary embodiment of the image reconstruction method, in operation S600, K-energy spectra of radiations are emitted toward the object (ob). According to an exemplary embodiment, radiation may be emitted toward the object (ob) by using a plurality of radiation emission units 100a and 100b, and radiation may be emitted toward the object (ob) by using one radiation emission unit 100. In addition, the K-energy spectrum of radiation may be emitted while rotating around the object (ob).

Subsequently, in operation S610, radiation emitted toward the object which has propagated through the object is detected, and measurement data corresponding to the detected radiation is acquired. In this case, the acquired measurement data may relate to and/or be determined by an energy spectrum of the emitted radiation, a type or a density of inner substances of the object (ob), and/or a distance at which radiation propagates through the inside of the object (ob). Values of the measurement data are expressible in accordance with the Equations 1 and 2 described above.

Then, in operations S620 and S621, an image reconstruction value is initialized. In particular, a predetermined initial value of the image reconstruction value is given, and an associated index i is set to be equal to 1. The image reconstruction value may relate to information which is associated with K+α inner substances of the object.

The predetermined initial value of the image reconstruction value may be randomly given by a user or system designer or randomly determined by the radiation imaging apparatus. The predetermined initial value of the image reconstruction value may relate to and/or be determined by a type of radiation imaging apparatus, the number or type of radiation energy spectra, a type of object (ob), an area of the object (ob) to be imaged, a type of radiation image to be acquired, and/or the like.

In operation S630, simulation data is calculated by using the initialized image reconstruction value. According to an exemplary embodiment, operation S630 may be omitted.

In operation S640, a correction value of the image reconstruction value is acquired. In one exemplary embodiment, the correction value of the image reconstruction value may be acquired by using the initialized image reconstruction value and/or simulation data. In this case, the correction value corresponds to a value which reduces and/or minimizes a discrepancy between the measurement data and the simulation data.

Subsequently, in operation S650, the image reconstruction value is updated by using the acquired correction value. In this case, the initial value is deleted and the correction value is recorded in the image reconstruction value field, thereby updating the image reconstruction value.

In operation S660, a determination is made regarding whether to repeat operations S630, S640, and S650. When the updated image reconstruction value, that is, the acquired correction value, is considered appropriate for image reconstruction (i.e., YES with respect to operation S660), a reconstructed image is acquired by using the acquired image reconstruction value in operation S670.

When the updated image reconstruction value is not appropriate for image reconstruction (i.e., NO with respect to operation S660), operations S630, S640, and S650 are repeated by using the acquired image reconstruction value to update the image reconstruction value and its associated index i in operation S661.

In an exemplary embodiment, the image reconstruction value may be continuously updated by repeating operations S630, S640, and S650 according to the number of repetitions predetermined by a user or a system designer. In another exemplary embodiment, the image reconstruction value may be repeatedly updated by repeating operations S630, S640, and S650 until the image reconstruction value reaches the predetermined value.

As a result, an accurate radiation image corresponding to the inherent inner substances of the object (ob) is reconstructed. For example, when measurement data is acquired by injecting three contrast agents into the object (ob) and emitting a plurality of (for example, three or more) different spectra of radiation thereto, a radiation image is reconstructed in which soft tissues and bones are clearly distinguished, in addition to the three contrast agents.

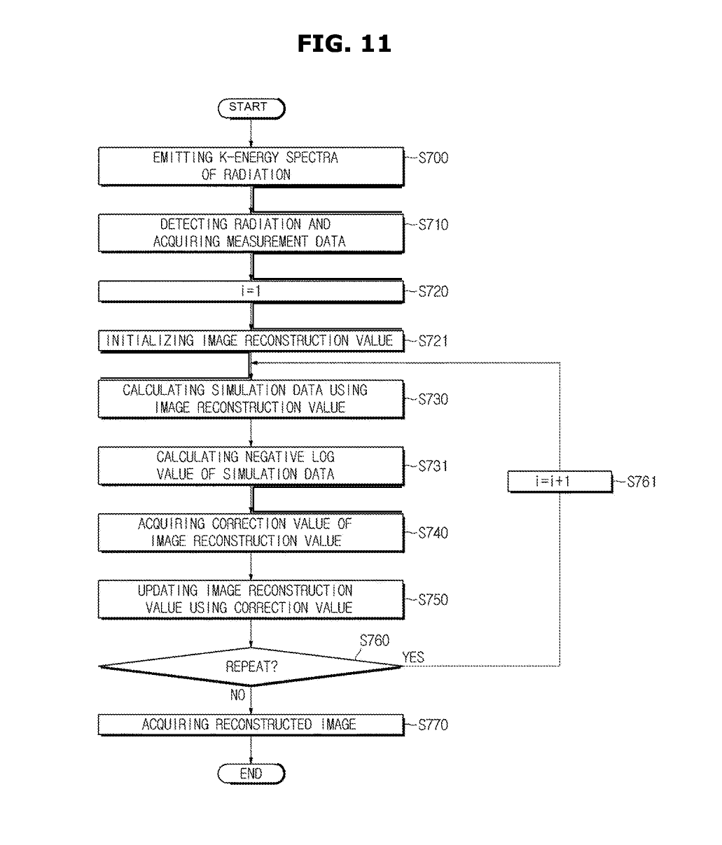

FIG. 11 is a flowchart illustrating an image reconstruction method, according to another exemplary embodiment.

As shown in FIG. 11, according to another exemplary embodiment of the image reconstruction method, in operation S700, K-energy spectra of radiation is emitted toward an object (ob), and then in operation S710, the emitted radiation is received and is detected, and predetermined measurement data corresponding to the detected radiation is acquired. As described above, the acquired measurement data may be related to and/or determined by at least one of an energy spectrum of the emitted radiation, a type and/or a density of inner substances of the object (ob), and/or a distance at which radiation propagates through the inside of the object (ob). Values of the measurement data are expressible in accordance with Equations 1 and 2 described above.

Next, in operations S720 and S721, a predetermined initial value is determined as the image reconstruction value and the image reconstruction value is initialized, and an associated index is set equal to 1. The image reconstruction value may relate to information which is associated with K+α substances of the inside of the object. As described above, the initial value of the image reconstruction value may be randomly determined and may be determined by imaging environment or purpose.

In operation S730, simulation data is calculated by using the initialized image reconstruction value, and in operation S731, a negative logarithmic value of the calculated simulation data is calculated. In particular, the negative log value of the simulation data is calculated by applying a negative logarithm to the simulation data.

In operation S740, a correction value of the image reconstruction value is acquired by using the negative log value of the simulation data. In this case, the correction value corresponds to a value which reduces and/or minimizes a discrepancy between the negative log value of the measurement data and the negative log value of the simulation data.

In operation S750, the image reconstruction value is updated by using the acquired correction value.

In operation S760, a determination is made regarding whether to repeat operations S730, S731, S740, and S750. When the updated image reconstruction value, that is, the acquired correction value is considered to be appropriate for image reconstruction (i.e., YES with respect to operation S760), an image is reconstructed by using the acquired image reconstruction value in operation S770.

When the updated image reconstruction value is considered inappropriate for image reconstruction (i.e., NO with respect to operation S760), in operation S761, the image reconstruction value and its associated index i are updated again by repeating operations S730, S731, S740, and S750 by using the acquired image reconstruction value. In this case, the number of repetitions may be determined by a user or a system designer or by the radiation imaging apparatus.

According to the radiation imaging apparatus and the image reconstruction method described above, more various inner substances of an object are separated when an inner structure of the object is observed using an imaging apparatus, and an accurate image corresponding to actual tissues of the object is thus reconstructed.

In addition, the radiation imaging apparatus reconstructs a radiation image in which more inner substances of an object than the number of types of energy spectra of radiations emitted to the object are distinguished from one another. Accordingly, it may be possible to obtain an effect that numerous types of substances are distinguished simply by emitting fewer types of energy spectra of radiation.

In addition, a radiation image having a high accuracy is reconstructed without deterioration in image quality, bones and contrast agents are clearly distinguished, and a radiation image of the inside of the object is thus more accurately reconstructed.

In addition, it may be possible to obtain an advantage of inhibiting or minimizing a generation of artifacts, such as, for example, beam hardening artifacts, with respect to the reconstructed image.

Although a few exemplary embodiments have been shown and described, it would be appreciated by those skilled in the art that changes may be made in these exemplary embodiments without departing from the principles and spirit of the present inventive concept, the scope of which is defined in the claims and their equivalents.

What is claimed is:

1. An image reconstruction method comprising:
emitting K-energy spectra of radiation toward an object and detecting K-energy spectra of radiation which propagate through the object in order to acquire measurement data which relates to an inside of the object;
initializing an image reconstruction value;
acquiring a correction value which relates to the image reconstruction value which correction value reduces a discrepancy between the acquired measurement data and simulation data, wherein the simulation data comprises data which is associated with an inside structure of the object which data is acquirable by using the initialized image reconstruction value; and
updating the image reconstruction value by using the acquired correction value, wherein the image reconstruction value relates to information which is associated with K+α substances of the inside of the object, wherein each of K and α is a respective natural number.

2. The image reconstruction method according to claim 1, further comprising repeating the acquiring the correction value by using the updated image reconstruction value and repeating the updating the image reconstruction value.

3. The image reconstruction method according to claim 1, wherein the image reconstruction value comprises information which is associated with a respective density of each of a plurality of substances of the inside of the object.

4. The image reconstruction method according to claim 1, wherein the acquiring the correction value comprises:
acquiring the correction value of the image reconstruction value which correction value reduces the discrepancy between the measurement data and the simulation data by minimizing a generalized information theoretic discrepancy (GID) function.

5. The image reconstruction method according to claim 4, wherein the generalized information theoretic discrepancy (GID) function is expressible in accordance with the following Equation 1:
$$G ( f ( r ) \vert \vert g ( r ) ) = \int f ( r ) ψ ( \frac{f ( r )}{g ( r )} ) d r \qquad [ Equation 1 ]$$
wherein ψ(v) is a convex function which reaches a minimal value when v is equal to 1 and satisfies Equation 2 below, and f(x) and g(x) are functions which are compared with each other regarding the discrepancy, and wherein Equation 2 is expressible as follows:
ρⱼ= [symbol] −Nⱼ⁻¹dⱼ. [Equation 2]

6. The image reconstruction method according to claim 4, wherein the acquiring the correction value comprises calculating the following Equation 2:
ρⱼ= [symbol] −Nⱼ⁻¹dⱼ [Equation 2]
wherein ρⱼ represents a density of a j^{th} voxel, \hat{p}ⱼ represents simulation data which relates to the density of the j^{th} voxel, Nⱼ represents a first adjustment value, and dⱼ represents a second adjustment value,
wherein the first adjustment value and the second adjustment value are values which are respectively derivable from at least one from among the generalized information theoretic discrepancy function and an approximation function which relates to the generalized information theoretic discrepancy.

7. The image reconstruction method according to claim 6, wherein the first adjustment value is expressible in accordance with the following Equation 3:
$${N}_{j} = {( {( \frac{\partial {x}_{j}}{\partial {ρ}_{j}} )}^{T} ( {\sum }_{i} {a}_{ij} {γ}_{i} {H}_{i}^{ɛ} ( ) ) ( \frac{\partial {x}_{j}}{\partial {ρ}_{j}} ) )}^{- 1} \qquad [ Equation 3 ]$$
wherein aᵢⱼ represents a level of contribution of the j^{th} voxel in a substance to an i^{th} pixel of a radiation detector,
$${γ}_{i} = {\sum }_{j} {a}_{ij}$$
is satisfied, Hᵢ^{ε}( [symbol] ) is a value which is derivable from at least one from among the generalized information theoretic discrepancy function and an approximation function which relates to the generalized information theoretic discrepancy,
$$\frac{\partial {x}_{j}}{\partial {ρ}_{j}}$$
is expressible in accordance with the following Equation 4, xⱼ represents a relative partial density of each substance included in the j^{th} voxel, f(x) is a function defining weights of α substances to be further reconstructed, ρ^{B} is a basis density of a substance to be virtually separated, N_{c} is K−1, I_{Nc} is an identity matrix, and Equation 4 is expressible as follows:
$$\frac{\partial {x}_{j}}{\partial {ρ}_{j}} = [ \begin{matrix}{ρ}_{j}^{B} \cdot \frac{\partial f ( {ρ}_{j}^{B} )}{\partial {ρ}_{j}^{B}} + f ( {ρ}_{j}^{B} ) & 0 \\ 0 & {I}_{Nc}\end{matrix} ] ∈ {R}^{( K = 1 ) \times K} . \qquad [ Equation 4 ]$$

8. The image reconstruction method according to claim 7, wherein the second adjustment value is expressible in accordance with the following Equation 5:
$${d}_{j} = ( {( \frac{\partial {x}_{j}}{\partial {ρ}_{j}} )}^{T} ( {\sum }_{i} {a}_{ij} {g}_{i}^{ɛ} ( ) ) \qquad [ Equation 5 ]$$
wherein gᵢ^{ε}( [symbol] ) is a value which is derivable from at least one from among the generalized information theoretic discrepancy function and an approximation function which relates to the generalized information theoretic discrepancy, pᵢ is a line projection of (K+α) substances, and [symbol] is a simulated line projection value of the (K+α) substances.

9. The image reconstruction method according to claim 8, wherein Hᵢ^{ε}( [symbol] ) is expressible in accordance with the following Equation 6 and gᵢ^{ε}( [symbol] ) is expressible in accordance with the following Equation 7:
$$\begin{matrix}{H}_{i}^{ɛ} ( ) = ( {e}_{i}^{3} {φ}^{″} ( {e}_{i} ) + {e}_{i}^{2} {φ}^{′} ( {e}_{i} ) ) {\sum }_{E} ( E ) M ( E ) & [ Equation 6 ] \\ {g}_{i}^{ɛ} ( ) = {e}_{i}^{2} {φ}^{′} ( {e}_{i} ) {\sum }_{E} ( E ) ( \frac{μ}{ρ} ) ( E ) & [ Equation 7 ]\end{matrix}$$
wherein eᵢ is expressible in accordance with the following Equation 8, M(E) is expressible in accordance with the following Equation 9, [symbol] (E) is expressible in accordance with the following Equation 10, μ is a linear attenuation, and sᵢ(E) is a spectrum of radiation which is emitted from a radiation emitter:
$$\begin{matrix}{e}_{i} = \frac{{y}_{i}}{} & [ Equation 8 ] \\ M ( E ) = ( \frac{μ}{ρ} ) \cdot {( \frac{μ}{ρ} )}^{T} & [ Equation 9 ] \\ ( E ) = {s}_{i} ( E ) exp ( - ( \frac{μ}{ρ} ) {( E )}^{T} {p}_{i} ) . & [ Equation 10 ]\end{matrix}$$

10. The image reconstruction method according to claim 8, wherein Hᵢ^{ε}( [symbol] ) is expressible in accordance with the following Equation 11 and gᵢ^{ε}( [symbol] ) is expressible in accordance with the following Equation 12:
$$\begin{matrix}{H}_{i}^{ɛ} ( ) = ( 2 {e}_{i} {φ}^{′} ( {e}_{i} ) + {e}_{i}^{2} {φ}^{″} ( {e}_{i} ) ) \times {\sum }_{E} \frac{{s}_{i}^{2} ( E ) M ( E )}{{t}_{i} ( E )} ( {e}_{i} + \frac{{β}_{i} ( E )}{{t}_{i} ( E )} ) & [ Equation 11 ] \\ {g}_{i}^{ɛ} ( ) = - {e}_{i} {φ}^{′} ( {e}_{i} ) {\sum }_{E} {s}_{i} ( E ) ( \frac{μ}{ρ} ) ( E ) ( {e}_{i} + \frac{{β}_{i} ( E )}{{t}_{i} ( E )} ) & [ Equation 12 ]\end{matrix}$$
wherein eᵢ is expressible in accordance with the following Equation 13, μ is a linear attenuation, sᵢ (E) is a spectrum of radiation emitted from a radiation emitter, tᵢ is expressible in accordance with the following Equation 14, βᵢ is a predetermined positive function wherein a marginal sum is equal to Bᵢ, in which Bᵢ is a predetermined constant, zᵢ is a value which is obtainable by calculating a negative logarithm (−log) of yᵢ, and ξᵢ(E) is a positive function, a sum of which satisfies Eᵢ:
$$\begin{matrix}{e}_{i} = \frac{( {z}_{i} + {E}_{i} )}{( + {E}_{i} )} & [ Equation 13 ] \\ {t}_{i} ( {p}_{i} , E ) = {s}_{i} ( E ) {μ ( E )}^{T} {p}_{i} + {ɛ}_{i} ( E ) . & [ Equation 14 ]\end{matrix}$$

11. The image reconstruction method according to claim 8, wherein Hᵢ^{ε}( [symbol] ) is expressible in accordance with the following Equation 15 and gᵢ^{ε}( [symbol] ) is expressible in accordance with the following Equation 16:
$$\begin{matrix}{H}_{i}^{ɛ} ( ) = \frac{1}{Y} ( 2 {e}_{i} {φ}^{′} ( {e}_{i} ) + {e}_{i}^{2} {φ}^{″} ( {e}_{i} ) ) \times {\sum }_{E} \frac{{s}_{i}^{2} ( E ) M ( E )}{{t}_{i} ( E )} ( \frac{U ( E )}{{t}_{i} ( E )} - {e}_{i} ) & [ Equation 15 ] \\ {g}_{i}^{ɛ} ( ) = \frac{1}{Y} {e}_{i} {φ}^{′} ( {e}_{i} ) {\sum }_{E} {s}_{i} ( E ) ( \frac{μ}{ρ} ) ( E ) ( {e}_{i} - \frac{U ( E )}{{t}_{i} ( E )} ) & [ Equation 16 ]\end{matrix}$$
wherein eᵢ is expressible in accordance with the following Equation 13, μ is a linear attenuation, sᵢ (E) is a spectrum of radiation emitted from a radiation emitter, tᵢ is expressible in accordance with the following Equation 14, and U(E) is a function, a sum of which satisfies Y:
$$\begin{matrix}{e}_{i} = \frac{( {z}_{i} + {E}_{i} )}{( + {E}_{i} )} & [ Equation 13 ] \\ {t}_{i} ( {p}_{i} , E ) = {s}_{i} ( E ) {μ ( E )}^{T} {p}_{i} + {ɛ}_{i} ( E ) . & [ Equation 14 ]\end{matrix}$$

12. A radiation imaging apparatus comprising:
a radiation emitter which is configured to emit multiple energy spectra of radiation toward an object;
a radiation detector which is configured to detect multiple energy spectra of radiation which propagate through the object and to output measurement data based on the detected multiple energy spectra; and
an image reconstructor which is configured to reconstruct a radiation image of the object, based on the outputted measurement data,
wherein the image reconstructor is further configured to acquire a correction value which relates to an image reconstruction value which correction value reduces a discrepancy between the measurement data and simulation data, and to update the image reconstruction value by using the acquired correction value,
wherein the simulation data includes data which relates to an inner structure of the object which is acquirable by using the image reconstruction value which relates to information which is associated with K+α substances of the inside of the object, wherein each of K and α is a respective natural number.

13. The radiation imaging apparatus according to claim 12, wherein the image reconstructor is further configured to initialize the image reconstruction value and to calculate the simulation data by using the initialized image reconstruction value.

14. The radiation imaging apparatus according to claim 12, wherein the image reconstructor is further configured to update the image reconstruction value at least one time.

15. The radiation imaging apparatus according to claim 12, wherein the image reconstruction value relates to information which is associated with a density of at least one substance of the inside of the object.

16. The radiation imaging apparatus according to claim 12, wherein the image reconstructor is further configured to acquire the correction value of the image reconstruction value by minimizing a generalized information theoretic discrepancy function.

17. An image reconstruction method comprising:
emitting a first energy spectrum of radiation and at least a second energy spectrum of radiation toward an object;
detecting a first energy spectrum of radiation and at least a second energy spectrum of radiation which propagate through the object in order to acquire measurement data which relates to an inside of the object;
initializing an image reconstruction value which is usable for reconstructing an image of the inside of the object;
using the initialized image reconstruction value to acquire simulation data which is associated with the inside of the object;
determining a correction value which relates to the image reconstruction value which correction value reduces a difference between the acquired measurement data and the acquired simulation data; and
updating the image reconstruction value by using the determined correction value.

18. The image reconstruction method according to claim 17, further comprising repeating the determining the correction value by using the updated image reconstruction value to acquire updated simulation data and repeating the updating the image reconstruction value with respect to the acquired updated simulation data.

19. The image reconstruction method according to claim 17, wherein the determining the correction value comprises optimizing a generalized information theoretic discrepancy (GID) function with respect to the acquired measurement data and the acquired simulation data.

20. A non-transitory computer-readable recording medium having recorded thereon a program which is executable by a computer for performing an image reconstruction method, the method comprising:
accessing measurement data which relates to an inside of an object which has been acquired as a result of a process which includes emitting K-energy spectra of radiation toward the object and detecting K-energy spectra of radiation which propagate through the object;
initializing an image reconstruction value;
acquiring a correction value which relates to the image reconstruction value which correction value reduces a discrepancy between the acquired measurement data and simulation data, wherein the simulation data comprises data which is associated with an inside structure of the object which data is acquirable by using the initialized image reconstruction value; and
updating the image reconstruction value by using the acquired correction value,
wherein the image reconstruction value relates to information which is associated with K+α substances of the inside of the object, wherein each of K and α is a respective natural number.
